(19) United States
(12) Patent Application Publication
(10) Pub. No.: US 2015/0209483 A1
(43) Pub. Date: Jul. 30, 2015

(54) BIOABSORBABLE MEDICAL DEVICES AND METHODS OF USE THEREOF

(71) Applicant: COOK MEDICAL TECHNOLOGIES LLC, Bloomington, IN (US)

(72) Inventors: Keith R. Milner, West Lafayette, IN (US); James M. Carlson, Warsaw, IN (US); Steven Charlebois, Lafayette, IN (US); Krista N. Gearhart, Lafayette, IN (US); Richard A. Swift, South Bend, IN (US)

(73) Assignee: COOK MEDICAL TECHNOLOGIES LLC, Bloomington, IN (US)

(21) Appl. No.: 14/683,486

(22) Filed: Apr. 10, 2015

Related U.S. Application Data
(63) Continuation of application No. 13/788,056, filed on Mar. 7, 2013.
(60) Provisional application No. 61/740,044, filed on Dec. 20, 2012.

(51) Int. Cl. A61L 31/08; A61L 31/02; A61L 31/14; A61F 2/82; A61L 31/16

(52) U.S. Cl. CPC A61L 31/08; A61F 2/82; A61L 31/16; A61L 31/148; A61L 31/022; A61F 2210/0004; A61F 2210/0076; A61L 2300/416; A61L 2300/606

(57) ABSTRACT

One aspect provides implantable medical devices including a bioabsorbable structure having a surface and having a coating layer on at least a portion of the surface. In certain embodiments, the coating layer includes a releasable bioactive and provides for a controlled absorption of the bioabsorbable structure upon implantation in a human or veterinary patient. Another aspect provides methods treating a disease including implanting such a device in a vessel of a human or veterinary patient.

### RELATED APPLICATIONS

This patent application is a continuation of U.S. patent application Ser. No. 13/788,056, filed Mar. 7, 2013, which claims the benefit of U.S. provisional patent application No. 61/740,044, filed Dec. 20, 2012, the entire contents of which applications are hereby incorporated by reference.

### TECHNICAL FIELD

The embodiments relate generally to human and veterinary implantable medical devices, at least a portion of which absorb when implanted. In certain embodiments, the devices have a coating incorporating a bioactive agent which controls the absorption of the device. The embodiments also provide methods of manufacturing and using such devices.

### BACKGROUND

It has become common to treat a variety of medical conditions by introducing an implantable medical device partly or completely into the esophagus, trachea, colon, biliary tract, urinary tract, vascular system or other location within a human or veterinary patient. For example, many treatments of the vascular system entail the introduction of a device such as a stent, catheter, balloon, wire guide, cannula, or the like. However, when such a device is introduced into and manipulated through the vascular system, the blood vessel walls can be disturbed or injured. Clot formation or thrombosis often results at the injured site, causing stenosis or occlusion of the blood vessel. Moreover, if the medical device is left within the patient for an extended period of time, a thrombus often forms on the device itself, again causing stenosis or occlusion. As a result, the patient is placed at risk of a variety of complications, including heart attack, pulmonary embolism and stroke. Thus, the use of such a medical device can entail the risk of precisely the problems that its use was intended to ameliorate.

A device such as an intravascular stent can be a useful adjunct to percutaneous transluminal angioplasty (PTA), particularly in the case of either acute or threatened closure after angioplasty. The stent is placed in the dilated segment of the artery to mechanically prevent abrupt closure and restenosis. Unfortunately, even when the implantation of the stent is accompanied by aggressive and precise antiplatelet and anticoagulation therapy (typically by systemic administration), the incidence of thrombotic vessel closure or other thrombotic complication remains significant, and the prevention of restenosis is not as successful as desired. Furthermore, an undesirable side effect of the systemic antiplatelet and anticoagulation therapy is an increased incidence of bleeding complications, most often at the percutaneous entry site.

Stents coated with a bioactive material such a paclitaxel, sirolimus or a sirolimus derivative have offered a means of overcoming such problems. Such devices deliver the bioactive material directly into a body portion during or following a medical procedure, so as to treat or prevent such conditions and diseases, for example, to prevent abrupt closure and/or restenosis of a body portion such as a passage, lumen or blood vessel. However, the use of drug-eluting stents presents some potential drawbacks. Such stents are typically formed from metal and may cause a number of complications. These include a predisposition to late stent thrombosis, prevention of vessel remodeling, inhibition of surgical revascularization and impairment of later medical imaging.

Bioabsorbable stents offer a means of overcoming some of these problems. These stents are typically formed of a bioabsorbable metal or polymer and degrade over time once implanted, thus eliminating the long-term use of antiplatelet therapy, without increasing the risk of stent thrombosis. In addition, bioabsorbable stents do not interfere with subsequent diagnostic imaging evaluations. However, the use of such stents may introduce additional problems, such as premature absorption of the stent structure resulting in stent collapse and blockage of the vessel.

### SUMMARY

One aspect of the present invention provides an implantable medical device including a bioabsorbable base material and a coating layer on at least a portion of the surface of the base material. The coating layer provides for a controlled absorption of the base material when the device is implanted. In certain embodiments, the bioabsorbable base material is a bioabsorbable metal, a bioabsorbable polymer or a mixture of these materials. In another embodiment, the structure is encapsulated (i.e. completely covered) by the coating layer.

In certain embodiments, the coating layer includes a bioactive, either alone or in combination with other material. In one embodiment, the bioactive controls the absorption of the base material when the device is implanted. The bioactive can be paclitaxel and can include dihydrate paclitaxel. In various embodiments, the coating layer reduces the absorption of the base material when the structure is implanted by at least 10% or 20% or 30%.

Another aspect of the present invention provides a method of locally delivering a bioactive agent within a body vessel. The method includes inserting an implantable device as described into the vascular system of a patient and radially expanding the medical device within the body vessel to bring tissue in contact with the device, delivering the bioactive agent to the tissue. In one embodiment, the expandable medical device is a vascular stent. In another embodiment, the coating layer includes dihydrate paclitaxel, which provides for a controlled absorption of the base material.

### BRIEF DESCRIPTION OF THE DRAWINGS

FIG. 1 shows a coated endoluminal medical device.

FIGS. 2 A-C show cross sectional views of embodiments of a portion of the medical device of FIG. 1. FIG. 2A shows an embodiment having a coating on the luminal, abluminal and side walls of the device. FIG. 2B shows an embodiment having a coating on the abluminal and side walls of the device. FIG. 2C shows an embodiment having a coating surrounding the abluminal and luminal walls of the device.

### DETAILED DESCRIPTION OF THE PREFERRED EMBODIMENTS

### Definitions

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

The term “bioabsorbable” is used herein to refer to materials that dissipate upon implantation within a body, independent of which mechanisms by which dissipation can occur, such as dissolution, degradation, absorption and excretion.

The term “adapted” for introduction into a human or veterinary patient is used herein to refer to a device having a structure that is shaped and sized for introduction into a human or veterinary patient.

As used herein, the term “body vessel” means any body lumen, including but not limited to blood vessels, esophageal, intestinal, biliary, urethral and ureteral passages.

The term “luminal surface,” as used herein, refers to the portion of the surface area of a medical device defining at least a portion of an interior lumen. Conversely, the term “abluminal surface,” refers to portions of the surface area of a medical device defining at least a portion of an exterior surface of the device. For example, where the medical device is a vascular stent having a cylindrical frame formed from a plurality of interconnected struts and bends defining a cylindrical lumen, the abluminal surface can include the exterior surface of the struts and bends, i.e. those portions of the struts and bends that are placed adjacent or in contact with the vessel wall when the stent is expanded, while the luminal surface can include the interior surface of the struts and bends, i.e. those portions of the struts and bends that are placed adjacent or in contact with the vessel interior when the stent is expanded.

The term “therapeutic effect” as used herein means an effect which induces, ameliorates or otherwise causes an improvement in the pathological symptoms, disease progression or physiological conditions associated with or resistance to succumbing to a disorder, for example restenosis, of a human or veterinary patient. The term “therapeutically effective amount” as used with respect to a therapeutic agent means an amount of the therapeutic agent which imparts a therapeutic effect to the human or veterinary patient.

### Implantable Medical Devices

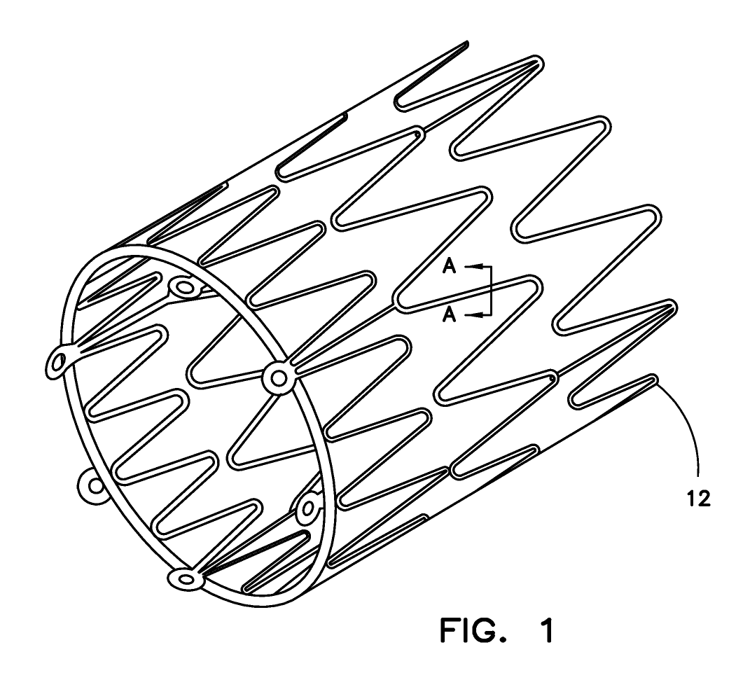

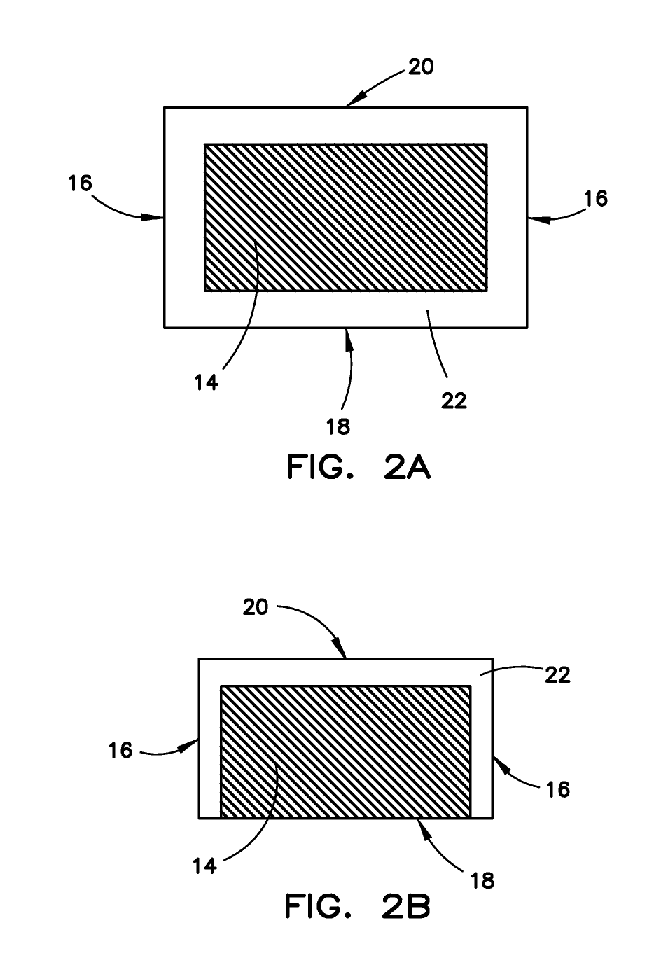

One aspect of the present invention provides an implantable medical device including a base structure having, at least, a bioadsorbable portion and a coating that controls the rate at which the bioabsorbable portion of the device absorbs after implantation. With reference now to FIG. 1, this figure shows an implantable medical device including a structure 12 adapted for introduction into a human or veterinary patient. For clarity, only a portion of the structure is shown. By way of example, structure 12 is configured as a self-expanding and force-expandable (e.g. balloon-expandable) vascular stent particularly adapted for insertion into the vascular system of the patient, such as into the coronary or peripheral vascular system. In other embodiments, the structure can be used in other systems and sites such as the esophagus, trachea, colon, biliary ducts, urethra and ureters among others. Indeed, the structure can alternatively be configured as any conventional vascular or other medical device, and can include any of a variety of conventional stents or other adjuncts.

The inserted structure need not be an entire device, but can merely be that portion of a vascular or other device which is intended to be introduced into the patient. Accordingly, the structure can be configured as at least one of, or any portion of, a catheter, a wire guide, a cannula, a stent, a vascular or other graft, an orthopedic device, appliance, implant, or replacement. The structure can also be configured as a combination of portions of any of these.

Preferably, however, the structure 12 is configured as a vascular stent. Such stents are typically about 10 to about 60 mm in length and designed to expand to a diameter of about 2 mm to about 6 mm when inserted into the vascular system of the patient. These stent dimensions are, of course, applicable to exemplary stents employed in the coronary arteries. Structures such as stents or catheter portions intended to be employed at other sites in the patient, such as in the aorta, peripheral vascular system, esophagus, trachea, colon, biliary tract, or urinary tract will have different dimensions more suited to such use. For example, aortic, esophageal, tracheal and colonic stents may have diameters up to about 25 mm and lengths about 100 mm or longer.

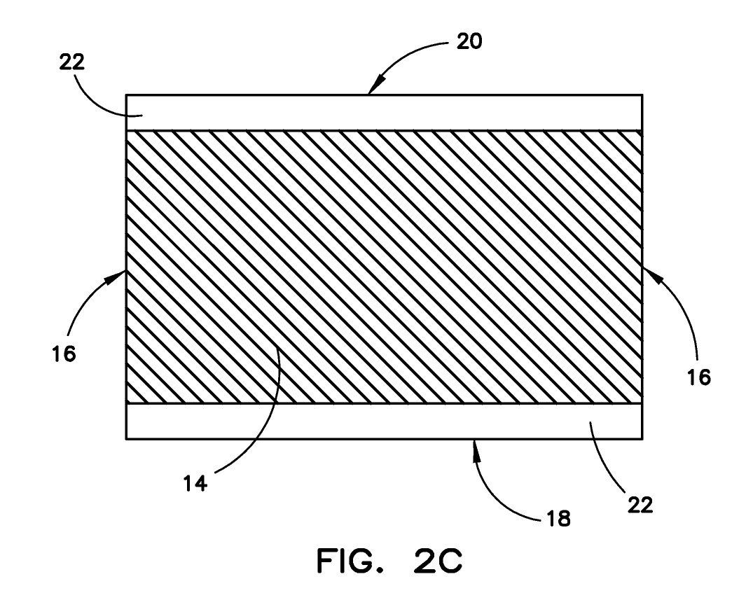

The structure 12 is at least partly formed from a bioabsorbable base material and is covered, at least in part, by a coating which controls the absorption of the base material after the device is implanted in the patient. FIGS. 2A-C illustrate cross sectional views across axis A-A′ of different embodiments of FIG. 1. FIG. 2A shows as embodiment having coating 22 surrounding luminal surface 18, abluminal surface 20 and side walls 16 of base structure 14. In the embodiment shown in FIG. 2B a coating is present on abluminal surface 20 and on and side walls 16 of the device but not on luminal surface 18. FIG. 2C shows an embodiment having a coating on luminal surface 18 and abluminal surface 20. In this embodiment, side walls 16 are free of a coating.

In certain embodiments, the entirety of the implantable portion of device is covered by a coating layer that controls the absorption of the base material once implanted. For example, in one embodiment, the device is a stent having such a coating on the entire luminal surface, abluminal surface and side walls. In other embodiments, a coating is present only on part of one or more of these surfaces. For example, an implantable stent can be provided with a coating on only portions of the stent by first masking portions of the device before applying the coating.

### Coating Methods

The coating can be applied to the medical device in any known manner. For example, a coating may be applied by spraying, dipping, pouring, pumping, brushing, wiping, vacuum deposition, vapor deposition, plasma deposition, electrostatic deposition, ultrasonic deposition, epitaxial growth, electrochemical deposition or any other method known to those skilled in the art.

In one embodiment, the coating material is dissolved in a solvent and sprayed onto the medical device under a fume hood using a spray gun, such as the Model Number 200 spray gun manufactured by Badger Air-Brush Company, Franklin Park, Ill. 60131. Alignment of the spray gun and medical device may be achieved with the use of laser beams, which may be used as a guide when passing the spray gun up and down the medical device being coated.

In another embodiment, the coating material is dissolved in a solvent and then sprayed onto the medical device using an electrostatic spray deposition (ESD) process. The ESD process generally depends on the principle that a charged particle is attracted towards a grounded target.

In yet another embodiment, the medical device is coated using an ultrasonic spray deposition (USD) process. Ultrasonic nozzles employ high frequency sound waves generated by piezoelectric transducers which convert electrical energy into mechanical energy. The transducers receive a high frequency electrical input and convert this into vibratory motion at the same frequency. This motion is amplified to increase the vibration amplitude at an atomizing surface. For example, the medical device can be coated using an ultrasonic spray nozzle, such as those available from Sono-Tek Corporation, Milton, N.Y. 12547.

### Bioabsorbable Implantable Devices

In certain embodiments, the base structure of the implantable medical devices includes certain metal materials that are bioabsorbable while still providing some of the advantages of mechanical durability provided by non-bioabsorbable metals. Certain devices, such as stents, can be formed from bioabsorbable metals or metal alloys that provide levels of radial flexibility desired from stent frames. For example, bioabsorbable metal stents can incorporate bioabsorbable materials such as magnesium, titanium, zirconium, niobium, tantalum, zinc, silicon, lithium, sodium, potassium, calcium, iron, manganese, yttrium, rare earth metals, such as neodymium, or alloys and/or mixtures of two or more of these materials. Some preferred metallic bioabsorbable material alloy compositions include lithium-magnesium, sodium-magnesium, zinc-titanium and alloys including magnesium in combination with at least one of yttrium, neodymium and zirconium. Further details of bioabsorbable metals useful in the manufacture of stent frames are described in U.S. Patent Publication Number 2010/0262221, the contents of which are incorporated by reference.

Other embodiments provide implantable devices including bioabsorbable polymers that absorb into the body after a period of time. A wide variety of bioabsorbable polymers can be used to form the device structure. Nonlimiting examples of bioabsorbable polymers include polyesters such as poly(hydroxyalkanoates), poly(lactic acid) or polylactide (PLA), poly(glycolic acid) or polyglycolide (PGA), poly(caprolactone), poly(valerolactone) and co-polymers thereof; polycarbonates; polyoxaesters such as poly(ethylene oxalate), poly(alkylene oxalates); polyanhydrides; poly(amino acids); polyphosphazenes; phosphorylcholine; phosphatidylcholine; various hydrogels; polydioxanone, poly(DTE carbonate), and co-polymers or mixtures of two or more of the above polymers. The implantable devices can also include various natural polymers such as fibrin, collagens, extracellular matrix (ECM) materials, dextrans, polysaccharides and hyaluronic acid.

In certain embodiments, the bioabsorbable portion of the base structure includes both at least one bioabsorbable metal and at least one bioabsorbable polymer. For example, a stent may be from at least one layer of polymer and at least one layer of bioabsorbable metal. Alternatively, the base structure may include bioabsorbable metal structures at least partially embedded in a bioabsorbable polymer.

In one embodiment, the coating, such as one of the coatings described above, reduces the rate of bioabsorption of at least a portion of the device after it is implanted. By reducing the rate at which the device absorbs, the coating can help maintain the structural integrity of the device over a longer period of time compared to a similar uncoated device. For example, if the device is a bioabsorbable stent, providing such a coating increases the period during which the stent frame maintains sufficient structural strength to support the vessel wall and prevent collapse of the vessel. In other embodiments, the coating allows the thickness of the stent to be decreased without reducing the period over which the stent maintains its structural strength.

The reduction of bioabsorption of the device is determined by measuring the weight loss of the device. The certain embodiments, the coating reduces the weight loss of the implanted portion of the device by 5, 7, 10, 15, 20, 30, 40, 50, 70, 90 or 100 percentage over a period of 30, 60, 100, 200, 300, 400 or 500 days compared to the weight loss of the same device without the coating. In other embodiments, the coating reduces the weight loss of the device by 150, 200, 250, 300 or 400 percentage over a period of 30, 60, 100, 200, 300, 400 or 500 days. In another embodiment, the reduction in bioabsorption is measured by determining the increase in time taken for 50 percentage of the device to absorb after the device is implanted. In various embodiments, the coating provides for an increase in time taken for the weight of the implantable portion of the device to decrease by 50 percentage by 5, 10, 20, 30, 50, 70, 100, 200, 300, 400 or 500 percentage.

A measure of the rate of absorption of the device when implanted can be obtained by an in vitro degradation test method, such as the test described in ASTM test standard F1635-11, the contents of which are incorporated by reference. Although this protocol provides a test method for degradable polymer resins and for surgical implants fabricated from such resins, the methods disclosed are applicable for estimating the rate of absorption of other bioabsorbable materials, such as bioabsorbable metal alloys.

For the purposes of determining a measure of the rate of absorption of the device when implanted in a subject, the device is incubated at 37 deg. C. in a closed container containing a phosphate buffered saline buffer and the amount of weight loss measured at the required time period(s.) Because the rate of absorption may depend on factors such as the mechanical load placed on the device and the fluid flow surrounding the test device, it is important that such factors are controlled. For the purposes of determining the rate of absorption of the device, the device is tested without a mechanical load and without fluid flow past the device.

The certain embodiments, the coating reduces the rate of weight loss of an mechanically unloaded device stored in a phosphate buffered saline buffer at 37 deg. C. by 5, 7, 10, 15, 20, 30, 40, 50, 70, 90 or 100 percentage over a period of 30, 60, 100, 200, 300, 400 or 500 days compared to the weight loss of the same device without the coating. In other embodiments, the coating reduces the rate of weight loss of a device tested under these conditions by 150, 200, 250, 300 or 400 percentage over a period of 30, 60, 100, 200, 300, 400 or 500 days. In other embodiments, the coating increases the time taken for the weight of the implantable portion of the device to decrease by 50 percentage by 5, 10, 20, 30, 50, 70, 100, 200, 300, 400 or 500 percentage.

### Bioactive Coated Devices

In certain embodiments, the coating on the bioabsorbable implantable device includes a bioactive that elutes from the device for delivery to the patient. For example, the coating may contain at least one of heparin or another thrombin inhibitor; hirudin or another antithrombogenic agent; urokinase or another thrombolytic agent; a fibrinolytic agent; a vasospasm inhibitor; a calcium channel blocker; nitric or another vasodilator; terazosin or another antihypertensive agent; an antimicrobial agent; an antibiotic; an antiplatelet agent; an antimitotic; a microtubule inhibitor; dimethyl sulfoxide; an actin inhibitor; a remodeling inhibitor; deoxyribonucleic acid; an antisense polynucleotide; methotrexate or another antiproliferative agent; tamoxifen citrate; a taxane agent, such as paclitaxel or a derivative thereof; a mammalian target of rapamycin (mTOR) inhibitor such as sirolimus or a derivative thereof such as pimecrolimus, tacrolimus, everolimus, zotarolimus, novolimus, myolimus, temsirolimus, deforolimus, or biolimus; an anti-cancer agent; dexamethasone or a dexamethasone derivative; an anti-inflammatory steroid or non-steroidal antiinflammatory agent; cyclosporin or another immunosuppressive agent; a peptide; a protein; an enzyme; an extracellular matrix component; a cellular component or another biologic agent; captopril; enalapril or another angiotensin converting enzyme (ACE) inhibitor; ascorbic acid; alpha tocopherol; superoxide dismutase; deferoxamine; an iron chelator or antioxidant; or mixtures of at least two of these agents.

The bioactive can be included in a layer also including a carrier material. For example, the bioactive can be present in a layer also including one or more bioabsorbable polymers, such as those mentioned above. In these embodiments, the polymer can exhibit properties that differ from those of the underlying structure. For example, the polymer can have a different absorption profile upon implantation.

In other embodiments, the coating does not include a carrier material, such as a polymer. In such embodiments, the bioactive material itself reduces the rate of bioabsorption of the device as described above. For example, the coating may include only the bioactive material or the bioactive material and other components that do not affect the bioabsorption of the base material. For the purposes of describing the present embodiments, the coating layer is considered to “consist essentially” of the bioactive material when it is free of other materials that affect the bioabsorption of the base material upon implantation.

In one embodiment, the bioactive material is a taxane or a taxane analogue or derivative, for example, paclitaxel. Taxene agents, including paclitaxel, are believed to disrupt mitosis (M-phase) by binding to tubulin to form abnormal mitotic spindles (i.e., a microtubule stabilizing agent) and can be used to mitigate or prevent restenosis. Additional details regarding taxane agents are described in U.S. Pat. No. 7,875,284 B2, the contents of which are incorporated by reference. An illustrative embodiment provides an implantable device, such as a stent, coated with paclitaxel such that the drug is eluted from the device over a certain time period after implantation.

Taxane therapeutic agent molecules, such as paclitaxel molecules, having the same molecular structure may be arranged in different solid forms that can be characterized and differentiated by one or more physical properties, including the rate of dissolution in various elution media (for example cyclodextrin or porcine serum). Once dissolved, the taxane therapeutic agent molecules having identical molecular structures but originating from different solid forms are indistinguishable in solution.

Solid forms of paclitaxel at room temperature include amorphous paclitaxel (“aPTX”), dihydrate crystalline paclitaxel (“dPTX”) and anhydrous crystalline paclitaxel. These different solid forms of paclitaxel can be characterized and identified using various solid-state analytical tools, for example as described by Jeong Hoon Lee et al., “Preparation and Characterization of Solvent Induced Dihydrate, Anhydrous and Amorphous Paclitaxel,” Bull. Korean Chem. Soc. v. 22, no. 8, pp. 925-928 (2001), incorporated herein by reference. For example, amorphous and dihydrate taxane solid forms may be readily identified and differentiated by visual appearance and elution rates in various elution media, such as Heptakis-(2,6-di-O-methyl)-beta-cyclodextrin or porcine serum as described in U.S. Publication Number 20080020013, the contents of which are incorporated by reference.

The dihydrate taxane solid form typically has an opaque white color, while the amorphous dihydrate taxane solid form typically has a clear transparent appearance. In addition, the presence of different solid forms of the taxane therapeutic agent in a medical device coating can be identified and quantified by contacting the coating with an elution medium that selectively dissolves one solid form more readily than a second solid form. In solution with an elution medium, such as porcine serum or blood, the presence of the taxane therapeutic agent can be identified, for example, by using ultraviolet (UV) spectroscopy or high pressure liquid chromatography (HPLC). In certain elution media such as porcine serum, the dihydrate taxane therapeutic agent structures dissolve more slowly than the amorphous and anhydrous solid forms. Additional details regarding taxane solid forms and the use of these forms in implantable devices are described in U.S. Pat. No. 7,875,284 B2, the contents of which are incorporated by reference.

On one embodiment, the device is coated with the crystalline dihydrate paclitaxel form only. In other embodiments, at least 99, 98, 95, 90, 85, 80, 75, 70, 65, 60, 50, 40, 30, 20, or 10 percentage of the paclitaxel coated onto the device is the dihydrate crystalline paclitaxel form. In certain embodiments, the paclitaxel is present at an amount of between 20 and 1 or 12 and 1 or 6 and 1 or 3 and 1 micrograms/mm² of the device surface. In other embodiments, the paclitaxel is present at approximately 20, 12, 6, 3, or 1 micrograms/mm² of the device surface.

Devices including different solid forms of other bioactives are also within the scope of the present invention. Non limiting examples of such bioactives include NSAIDs, such as indomethacin; steroids, such as prednisolone; statins, such as atorvastatin; antimitotics, such as griseofulvin; antihyperlipidemics, such as probucol and immunosuppressants, such as rapamycin.

### Methods of Delivery and Treatment

Another aspect of the invention provides a method of treatment involving inserting into a patient or non-human subject an implantable medical device having any of the novel configurations described above and delivering a therapeutically effective amount of the bioactive agent as described above to the body of the patient or non-human subject.

For example, when the implantable medical device is a vascular stent coated as described above, the method of treatment involves implanting the stent into the coronary or peripheral vascular system of a patient and allowing a therapeutically effective amount of the bioactive agent(s) to be released from the stent in a controlled manner to treat a condition such as restenosis. In certain embodiments, the bioactive agent is a taxane, a taxane analogue or a derivative thereof, for example paclitaxel. In other embodiments, the bioactive agent is sirolimus or a derivative thereof, such as pimecrolimus, tacrolimus, everolimus, zotarolimus, novolimus, myolimus, temsirolimus, deforolimus, or biolimus. In one preferred embodiment, the coated medical devices are implanted to treat peripheral vascular disease by implanting the coated medical device in a peripheral artery.

The certain embodiments, a coating is provided that reduces the bioabsorption of the device by 5, 7, 10, 15, 20, 30, 40, 50, 70, 90 or 100 percentage, over a period of 30, 60, 100, 200, 300, 400 or 500 days, compared to the bioabsorption of the same device without the coating. In other embodiments, the coating reduces the bioabsorption of the device by 150, 200, 250, 300 or 400 percentage over a period of 30, 60, 100, 200, 300, 400 or 500 days. In yet other embodiments, the coating increases the time taken for the weight of the implantable portion of the device to decrease by 50 percentage by 5, 10, 20, 30, 50, 70, 100, 200, 300, 400 or 500 percentage.

The dosage level and period of release of the bioactive agent may be tailored to the subject being treated, the severity of the affliction, the judgment of the physician, and the like. In one embodiment of the invention, a vascular stent is coated with a drug at a concentration of 0.1-4 micrograms/mm². In another embodiment, the stent is coated with a drug at a concentration of 0.1-2 micrograms/mm². In yet another embodiment, the stent is coated with a drug at a concentration of 0.1-1 micrograms/mm².

### EXAMPLES

### Example 1 (Prophetic)

### Ultrasonic Spray Coating of Stents with Paclitaxel

Stents with coatings of paclitaxel taxane therapeutic agent including the dihydrate solid form of paclitaxel are prepared by spray coating a solution including paclitaxel, methanol and water. A paclitaxel solution in methanol and water is prepared using 68% methanol by dissolving about 8 mg of paclitaxel in 5 mL of previously made solution of 68% methanol 32% water. The solution is sprayed from an ultrasonic spray gun (Sono-tek Model 06-04372) in a glove box. Before spraying, the glove box is purged with nitrogen at 20 psi for 15 minutes. The atmosphere in the glove box was adjusted until the oxygen meter reads a constant 200 ppm within the glove box. The temperature in the glove box is set to 31° C. (88° F.).

The paclitaxel solution is loaded into a syringe and placed on a syringe pump in the ultrasonic coating apparatus and an absorbable metal stent (Mg alloy—WE43B) is mounted on a mandrel aligned with the spray nozzle. The solution is sprayed onto the stent using the spray gun.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

What is claimed is:

1. An implantable medical device comprising:
a structure having a surface and comprising a bioabsorbable base material; and
a coating layer on at least a portion of the surface,
wherein the coating layer provides for a controlled absorption of the base material.

2. The implantable medical device of claim 1, wherein the bioabsorbable base material is selected from the group consisting of a bioabsorbable metal, a bioabsorbable polymer and a mixture thereof.

3. The implantable medical device of claim 2, wherein the bioabsorbable base material is a bioabsorbable metal.

4. The implantable medical device of claim 1, wherein the device is a vascular stent.

5. The implantable medical device of claim 4, wherein the structure is encapsulated by the coating layer.

6. The implantable medical device of claim 1, wherein the coating layer comprises a bioactive.

7. The implantable medical device of claim 6, wherein the coating layer consists essentially of a bioactive.

8. The implantable medical device of claim 7, wherein the bioactive is paclitaxel.

9. The implantable device of claim 8, wherein the paclitaxel comprises dihydrate paclitaxel.

10. The implantable medical device of claim 1, wherein the coating layer reduces the absorption of the base material when the structure is implanted by at least 10% over a period of 100 days.

11. The implantable medical device of claim 10, wherein the coating layer reduces the absorption of the base material when the structure is implanted by at least 20% over a period of 100 days.

12. The implantable medical device of claim 11, wherein the coating layer reduces the absorption of the base material when the structure is implanted by at least 30% over a period of 100 days.

13. A method of locally delivering a bioactive agent within a body vessel, comprising
inserting an expandable medical device into the vascular system of a patient, the device comprising (i) a structure having a surface and comprising a bioabsorbable base material, and (ii) a coating layer on at least a portion of the surface, wherein the coating layer provides for a controlled absorption of the base material,
radially expanding the medical device within the body vessel to bring a vessel wall in contact with the device, and
maintaining the expanded device in contact with the vessel wall for a time sufficient to deliver a therapeutically effective amount of the bioactive agent to the vessel wall.

14. The method of claim 13, wherein the wherein the bioactive is paclitaxel.

15. The method of claim 14, wherein the paclitaxel comprises dihydrate paclitaxel.

16. The method of claim 13, wherein the coating layer reduces the absorption of the base material when the structure is implanted by at least 10% over a period of 100 days.

17. The method of claim 13, wherein the coating layer reduces the absorption of the base material when the structure is implanted by at least 20% over a period of 100 days.

18. The method of claim 13, wherein the expandable medical device is a vascular stent.

19. The method of claim 13, wherein the bioabsorbable base material is a metal or a metal alloy.

20. An implantable stent comprising:
a structure having a surface and comprising a bioabsorbable metal; and
a coating layer encapsulating the surface,
wherein the coating layer comprises dihydrate paclitaxel and provides for a controlled absorption of the base material.
